(19) United States
(12) Patent Application Publication
(10) Pub. No.: US 2026/0354874 A1
(43) Pub. Date: Nov. 12, 2026

(54) Orthopedic Implant In The Form Of A Plate To Be Fixed Between Two Bone Parts

(71) Applicant: Stryker European Operations Holdings LLC, Portage, MI (US)

(72) Inventors: Bernard Prandi, Rennea (FR); Keith Wapner, Philadelphia, PA (US); Charles P. Wapner, Media, PA (US); Peter W. Wapner, Media, PA (US)

(21) Appl. No.: 19/639,887

(22) Filed: Apr. 6, 2026

Related U.S. Application Data
(63) Continuation of application No. 19/025,037, filed on Jan. 16, 2025, now Pat. No. 12,616,507.
Application No. 19/025,037 is a continuation of application No. 17/990,936, filed on Nov. 21, 2022, now Pat. No. 12,521,155.
Application No. 17/990,936 is a continuation of application No. 16/429,834, filed on Jun. 3, 2019, now Pat. No. 11,534,212.
Application No. 16/429,834 is a continuation of application No. 15/130,147, filed on Apr. 15, 2016, now Pat. No. 10,349,988.
Application No. 15/130,147 is a continuation of application No. 14/734,676, filed on Jun. 9, 2015, now Pat. No. 9,333,013.
Application No. 14/734,676 is a continuation of application No. 14/041,706, filed on Sep. 30, 2013, now Pat. No. 9,078,713.
Application No. 14/041,706 is a continuation of application No. 12/918,071, filed on Oct. 2, 2009, now Pat. No. 8,556,946.

(30) Foreign Application Priority Data
Oct. 2, 2008 (FR) 0856694

(51) Int. Cl. A61B 17/80; A61B 17/68; A61B 17/84

(52) U.S. Cl. CPC A61B 17/8014; A61B 17/8004; A61B 17/8052; A61B 17/8057; A61B 17/8061; A61B 17/808; A61B 17/809; A61B 17/846; A61B 2017/681

(57) ABSTRACT

The invention relates to a plate fixed between two bone parts by way of screws engaged in holes formed in the thickness of said plate. The plate comprises an angled member or rib which is inclined according to an angle of between about 30° and 60° in relation to the plane defined by the plate. The angled member or rib has a hole for engaging a screw and is located in the central part of the width, over a determined part of the length of the plate, so that the screw brings the two bone parts into a compressive position.

### CROSS-REFERENCE TO RELATED APPLICATIONS

This application is a continuation of U.S. application Ser. No. 19/025,037, filed on Jan. 16, 2025, which is a continuation of U.S. application Ser. No. 17/990,936, filed Nov. 21, 2022, now U.S. Pat. No. 12,521,155, which is a continuation of U.S. application Ser. No. 16/429,834, filed Jun. 3, 2019, and now U.S. Pat. No. 11,534,212, which is a continuation of U.S. application Ser. No. 15/130,147, filed Apr. 15, 2016, and now U.S. Pat. No. 10,349,988, which is a continuation of U.S. application Ser. No. 14/734,676, filed Jun. 9, 2015, and now U.S. Pat. No. 9,333,013, which is a continuation of U.S. application Ser. No. 14/041,706, filed Sep. 30, 2013, and now U.S. Pat. No. 9,078,713, which is a continuation of U.S. application Ser. No. 12/918,071, filed Oct. 29, 2010 and now U.S. Pat. No. 8,556,946, which is a national phase entry under 35 U.S.C. § 371 of International Application No. PCT/FR2009/051879, filed Oct. 2, 2009, published in French, which claims priority from French Patent Application No. 0856694, filed Oct. 2, 2008, all of which are incorporated herein by reference in their entireties.

### BACKGROUND OF THE INVENTION

The invention relates to the technical field of orthopedic implants.

More particularly, the invention relates to a plate for arthrodesis or osteosynthesis adapted to be fixed between two bone parts.

In a manner known to one having ordinary skill in the art, this type of plate generally has holes for engaging screws, allowing arthrodesis between two bones or osteosynthesis between two bone fragments. This is, for example, the case for bones of the hand or foot, without however excluding other applications, particularly in the field of the spine. Depending on the pathology to be treated, these plates can have a general rectilinear or other geometric shapes.

From this state of the art, one of the objects the invention proposes to attain is to improve, in a sure and efficient manner, compression in a precise direction between the bone parts subjected to the plate.

To attain the given object to enhance the compression between the two relative bone parts, according to the invention, the plate has a formation that orients at least one screw at an angle with respect to a plane defined by the plate, the angle being between about 30° and 60°.

According to an advantageous embodiment, the formation is a tab that is angled according to an angle between 30° and 60°, and having a hole for engaging the screw. The angled tab results from a cut out and a deformation of a portion of the plate.

In another embodiment, the formation is a hole angled at an angle between 30° and 60° for engaging the screw.

Considering the problem to be solved, the formation is located on a determined portion of the length of the plate so that the screw ensures the compression of the two bone parts.

### BRIEF DESCRIPTION OF THE DRAWINGS

The invention is described hereinafter in more detail, with reference to the attached drawings in which:

FIG. 1 is a perspective view of an embodiment of the plate;

FIG. 2 is a side view of the plate;

FIGS. 3 and 4 are perspective views showing the mounting of the plate between two bone parts and their orientation by means of the plate according to the invention, the bone parts being shown schematically.

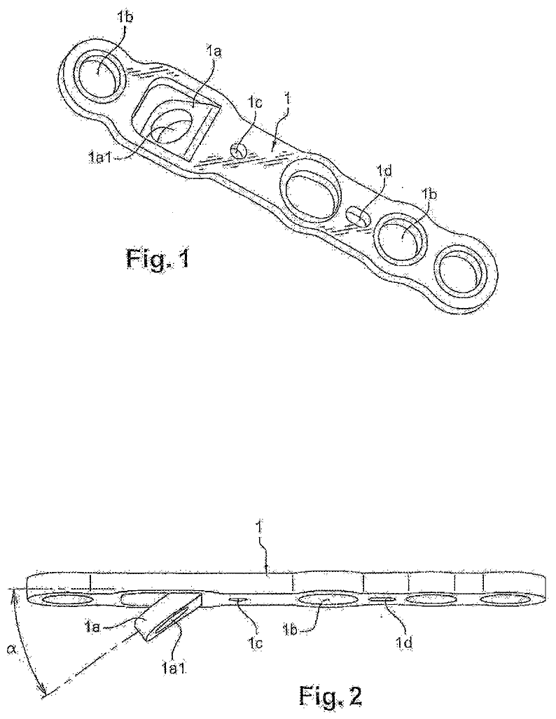

### DETAILED DESCRIPTION

According to the invention, the plate 1 has at least one formation 1a adapted to enable the positioning of at least one screw 2, at an angle α of between 30° and 60° with respect to a plane of the plate (FIG. 2).

In one embodiment, the formation 1a is an angled tab cut out and deformed from the plate. For example, the deformation is made with a cutting-punching operation. This angled tab has a hole 1a1 for screw 2. The angled tab 1a is positioned along the length of the plate so that after the screw 2 is fitted to it, the screw ensures the compression together of the two bone parts, as indicated below in the description.

In another embodiment, to allow for an angular orientation of the screw 2 according to an angle between about 30° and 60°, the formation 1a can be formed as an angled hole. It must be noted that the tab 1a enables adaptation of the angle as a function of the pathology to be treated, given that it is possible to deform this tab at will. In other words, the angle can be adjusted over a few degrees directly by the surgeon in the operating room, using an appropriate tool.

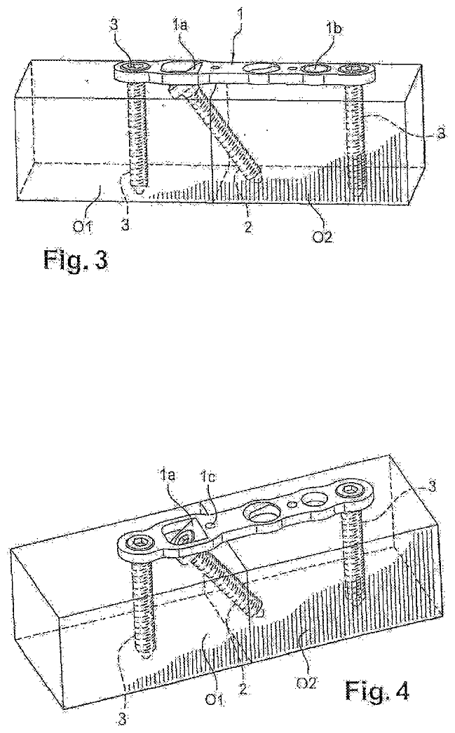

With reference to FIGS. 3 and 4 that show the positioning of the plate 1 between two bone parts O1 and O2:

Once the osteotomies have been carried out, a template of the plate, which does not have a guide formation, enables the position of the tab to be determined.

After determining the position of the tab, the surgeon makes a corresponding recess with the appropriate rasp.

Once the plate having the tab has been positioned, the surgeon sets one or two screws 3, on a side of the site of the osteosynthesis or the arthrodesis toward the tab. A temporary fastening pin can, possibly, be positioned in a complementary lug.

The screw 2 is then engaged in the hole 1a1 of the tab 1a to place the fracture in compression.

Once the compression has been done, the surgeon can screw one or several other additional fastening screws 3 and remove the temporary pin.

In a known manner, this plate 1 has smooth and/or threaded holes for the fastening screws 3 set in the bone parts O1 and 02 to engage in, as shown in FIGS. 3 and 4.

Similarly, the plate 1 can have at least one hole 1c for a pin for temporarily positioning the plate 1. Advantageously, the plate 1 can have a guide 1c for the insertion of a pin on the side of one of the bone parts O1 and another guide 1d for the insertion of another pin on the side of the other bone part O2.

Considering the effect of the desired compression, such as indicated above, the guide 1c is a circular hole whose diameter corresponds substantially to that of the pin, whereas the other guide 1d can be an elongated slot.

These provisions thus enable the bone to slide under the plate 1 as the screws are set, while ensuring compression along a precise direction, generally axially or parallel to the plate. The pins are of any known and appropriate type, and perfectly known to one having ordinary skill in the art.

The plate 1 can have several shapes, so that the holes 1a in particular can be aligned or arrayed, all or in part, according to the corners of a triangle or of a quadrilateral. These provisions, in triangle or in quadrilateral, of the screws, improve the stability of the mounting.

It must be noted also that the plate 1, no matter its shape, can be longitudinally bent so as to adapt to the curvature of the bone, consequently enabling the screws to form an angle between them.

The advantages are readily apparent from the description.

1. A bone plate for fusing first and second bone parts, the bone plate comprising:
a monolithic body having a proximal surface, an opposite distal bone-contacting surface, and a length extending along a longitudinal axis across the first and second bone parts;
a first hole and a second hole extending through the body and spaced apart along the longitudinal axis, the first hole positioned on the first bone part and configured to receive a first fixation member, the second hole positioned on the second bone part and configured to receive a second fixation member;
a tab formed integrally with the body, the tab extending downward beyond the distal bone-contacting surface at an angle of between about 30° and 60° relative to a plane of the body, the tab having a through-hole configured to receive a third fixation member having a head; and
a guide slot extending through the body between the proximal and distal surfaces and located directly above the tab,
wherein the guide slot defines a perimeter substantially similar to a perimeter of the tab, a central axis of the through-hole of the tab being angled to cross between the first and second bone parts and to pass through the guide slot, the guide slot being dimensioned to pass the head of the third fixation member therethrough and into the through-hole.

2. The bone plate of claim 1, wherein the tab is joined to the body along a single fold edge and cantilevered therefrom, such that a free edge of the tab opposite the single edge is spaced apart from both the proximal surface and the distal bone-contacting surface.

3. The bone plate of claim 2, wherein the guide slot is open along three sides corresponding to cut edges of the tab, the fold edge defining a fourth side of the guide slot.

4. The bone plate of claim 2, wherein the tab is plastically deformable about the fold edge to adjust the angle of the central axis of the through-hole relative to the plane.

5. The bone plate of claim 1, wherein a gap is defined between an underside of the body adjacent the guide slot and the tab, the gap being dimensioned to receive a shank of the third fixation member as the third fixation member passes from the guide slot into the through-hole.

6. The bone plate of claim 1, wherein the through-hole has a diameter smaller than the head of the third fixation member, the tab defining a seat surface engageable with the head to seat an entirety of the head below the proximal surface.

7. The bone plate of claim 1, wherein the guide slot, the through-hole, and at least one of the first and second holes are aligned with one another along the longitudinal axis.

8. The bone plate of claim 1, further including a first pin hole with a diameter substantially equal to a diameter of a guide pin, and a second pin hole defining an elongate slot extending lengthwise along the longitudinal axis to permit a guide pin received therein to translate along the longitudinal axis during compression of the first and second bone parts.

9. The bone plate of claim 8, wherein the first pin hole has an axis directed into the first bone part and the second pin hole has an axis directed into the second bone part.

10. The bone plate of claim 1, wherein the first and second holes are threaded locking holes and the through-hole is non-threaded.

11. A surgical kit for fusing a first bone part and a second bone part, the kit comprising:
a bone plate having a proximal surface, a distal bone-contacting surface, a body spanning the first and second bone parts, and an angled member extending downward beyond the distal bone-contacting surface, the angled member having a hole configured to receive a fastener oriented to cross from the first bone part to the second bone part;
a template defining an outline of the bone plate, the template having a locating feature positioned to indicate a location of the angled member; and
a bone-removal instrument having a working end profiled to form in at least one of the first and second bone parts at the indicated location a recess complementary to and configured to receive the angled member.

12. The kit of claim 11, wherein the bone-removal instrument is a rasp.

13. The kit of claim 11, wherein the working end of the bone-removal instrument defines a profile substantially matching the angled member such that the recess receives the angled member with the distal bone-contacting surface seated against bone.

14. The kit of claim 11, wherein the locating feature of the template includes an aperture aligned with the location of the angled member.

15. The kit of claim 11, wherein the template is substantially planar and lies flat against bone.

16. The kit of claim 11, further including the fastener, the fastener being a compression screw having a head, the hole of the angled member having a diameter smaller than the head such that an entirety of the head is seatable below the proximal surface.

17. The kit of claim 11, wherein the bone plate further includes a first locking hole and a second locking hole on opposite sides of the angled member, the kit further includes a first bone screw receivable in the first locking hole and a second bone screw receivable in the second locking hole.

18. The kit of claim 11, further including a first guide pin and a second guide pin, the bone plate including a circular pin hole sized to receive the first guide pin and an elongate slot sized to receive the second guide pin to permit the second guide pin to translate within the elongate slot during compression of the first and second bone parts.

19. The kit of claim 11, wherein the angled member is a tab formed integrally with and bent from the bone plate, the bone plate defining a guide slot extending through the bone plate directly above the tab and dimensioned to pass a head of the fastener into the hole of the angled member.

20. The kit of claim 11, wherein the bone plate is contoured to anatomically fit bones in a human foot, and the angled member is angled at between about 30° and 60° relative to a plane of the bone plate.
